(19) United States
(12) Patent Application Publication
(10) Pub. No.: US 2012/0101392 A1
(43) Pub. Date: Apr. 26, 2012

(54) OPTIMIZATION OF ARRHYTHMIA DETECTION BASED ON PATIENT ACTIVITY

(75) Inventors: Sourav K. Bhunia, Shoreview, MN (US); Xusheng Zhang, Shoreview, MN (US)

(73) Assignee: Medtronic, Inc.

(21) Appl. No.: 12/912,111

(22) Filed: Oct. 26, 2010

(51) Int. Cl. A61B 5/0205

(52) U.S. Cl. 600/483

(57) ABSTRACT

An implantable medical device is configured to perform a method for detecting cardiac events by sensing a patient activity signal and determining a patient activity trend in response to the activity signal. A cardiac event detection threshold is set in response to the patient activity trend. The device senses a cardiac signal and detects a cardiac event in response to the cardiac signal and the detection threshold.

### TECHNICAL FIELD

This disclosure relates generally to implantable medical devices for monitoring patients and, in particular, to a method and apparatus optimizing arrhythmia detection based on patient activity.

### BACKGROUND

Implantable medical devices (IMDs), such as implantable pacemakers and implantable cardioverter defibrillators (ICDs), utilize a variety of techniques and/or algorithms to detect heart arrhythmias. Ventricular arrhythmias, such as ventricular fibrillation (VF) and hemodynamically unstable ventricular tachycardia (VT), are particularly dangerous, and can result in death if not quickly terminated by delivery of a therapy. Consequently, IMDs are programmed to deliver therapy, such as defibrillation or cardioversion shocks, upon detecting VF or unstable VT.

IMDs typically detect VF and VT by measuring the intervals between ventricular depolarizations, i.e., ventricular cycle lengths (VCLs). A sustained period of short VCLs can be detected as VF or unstable VT when a required number of intervals falling within a defined VCL range or zone defined for the respective arrhythmia are counted. The minimum number of intervals required for detecting a particular arrhythmia is commonly referred to as the “number of intervals to detect” or “NID”.

Supraventricular tachycardias (SVTs), such as sinus tachycardia, atrial fibrillation, atrial flutter, and reentrant atrial tachycardia, can be conducted to the ventricles, and can lead to short VCLs that falsely indicate VF or VT. Delivery of defibrillation or cardioversion shocks to the ventricles in situations where an SVT causes VF or VT to be falsely detected is generally not clinically needed and may not be effective in terminating the SVT. Depending on the programmed value of the NID, a non-sustained tachyarrhythmia may be detected and responded to with an unneeded cardioversion or defibrillation therapy. Defibrillation and cardioversion shocks, which may be delivered repeatedly during an episode that has been inappropriately detected as a treatable VF of VT, can cause significant patient discomfort or induce a VF or VT.

In order to avoid inapproppriate detection of VF or VT during SVT episodes, some IMDs apply further analysis of the ventricular rhythm and may additionally analyze the atrial rhythm. If the criteria for VF or VT is met during a particular ventricular interval and the further analysis indicates the presence of an SVT, an IMD typically avoids detection of VF or VT during that interval and, in some cases, delivers a therapy to one or more atria, such as anti-tachycardia pacing or a cardioversion shock. Nonetheless, because IMDs are programmed to err on the side of over-detecting potentially lethal VF and VT, IMDs occasionally inappropriately detect VF or VT when the fast ventricular rhythm is non-lethal, e.g., caused by an SVT or non-sustained VT, despite such additional analysis.

In order to avoid inappropriate delivery of defibrillation and cardioversion shocks, and the substantial patient discomfort associated therewith, clinicians in some cases program IMDs to prevent delivery of therapy for ventricular rhythms that are generally slower than a certain cycle length. Slow arrhythmias can cause patient symptoms, however, such as fatigue, dizziness, and fainting, and can quickly accelerate into a more dangerous arrhythmia. Further, such clinician programming generally does not eliminate all inappropriate detection of VF and VT. Accordingly, an unmet need remains for improving the accurate detection of treatable ventricular arrhythmias.

### BRIEF DESCRIPTION OF THE DRAWINGS

FIG. 1 is a conceptual diagram illustrating an example of an IMD that is capable of providing arrhythmia therapies along with monitoring patient physical activity.

FIG. 2 is a functional block diagram of an IMD.

FIG. 3 is a flow chart of a method for controlling arrhythmia detection based on patient activity.

FIG. 4 is a flow chart of an alternative method for controlling arrhythmia detection parameters based on patient activity.

FIG. 5 is a flow chart of one method for adjusting NID in response to a patient's activity level and activity trend.

FIG. 6 is a time line illustrating the behavior of an arrhythmia detection parameter over time as patient activity changes.

FIG. 7 is a time line depicting adjustment of NID based on a delay time following detection of an increasing activity.

### DETAILED DESCRIPTION

In the following description, references are made to illustrative embodiments. It is understood that other embodiments may be utilized without departing from the scope of the disclosure. In some instances, for purposes of clarity, identical reference numbers may be used in the drawings to identify similar elements.

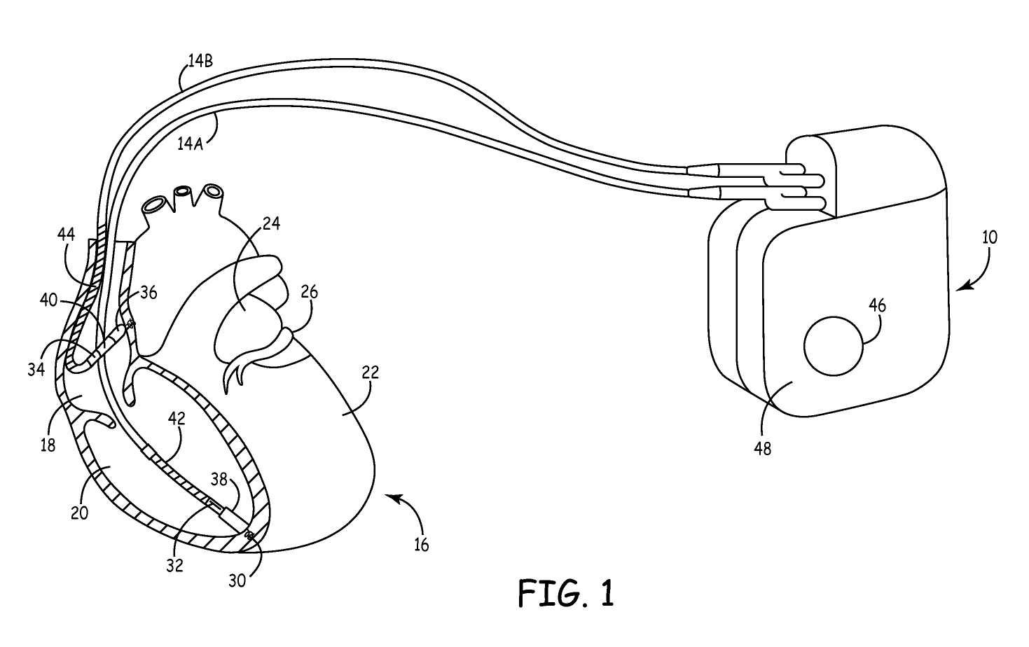

FIG. 1 is a conceptual diagram illustrating an example of an IMD 10 that is capable of providing arrhythmia therapies along with monitoring patient physical activity. IMD 10 may be embodied as a multi-chamber ICD having pacing, cardioversion and/or defibrillation capability. In the embodiment illustrated in FIG. 1, IMD 10 is coupled to leads 14A and 14B (collectively “leads 14”) that extend into the heart 16 of a patient. More particularly, right ventricular (RV) lead 14A extends through one or more veins (not shown), the superior vena cava, and right atrium 18, and into right ventricle 20, and right atrial (RA) lead 14B extends through the veins and superior vena cava, and into the right atrium 18 of heart 16.

IMD 10 senses electrical signals attendant to the depolarization and repolarization of heart 16 and provides pacing pulses via electrodes located on leads 14. Each of leads 14 includes a number of insulated electrical conductors extending from a proximal lead connector to each electrode carried by the leads. Located adjacent to the distal end of leads 14A and 14B are electrodes 30 and 32, and electrodes 34 and 36, respectively. Electrodes 32 and 34 take the form of ring electrodes, and tip electrodes 30 and 36 may take the form of extendable helical tip electrodes mounted retractably within insulative electrode heads 38 and 40, respectively. Each of the electrodes 30-36 is coupled to one of the insulated conductors within the lead body of its associated lead 14 and further coupled to circuitry within housing 48 of IMD 10.

Sense/pace electrodes 30-36 may be selected in unipolar or bipolar configurations to sense electrical signals attendant to the depolarization and repolarization of heart 16. The electrical signals are conducted to IMD 10 via leads 14. At least some of sense/pace electrodes 30-36 may further deliver pacing to cause depolarization of cardiac tissue in the vicinity thereof. IMD 10 may include one or more housing electrodes, such as housing electrode 46, formed integrally with an outer surface of IMD housing 48. In such embodiments, any of electrodes 30-36 are capable of being used for unipolar sensing or pacing in combination with housing electrode 46.

Leads 14A and 14B also carry elongated coil defibrillation electrodes 42 and 44, respectively. IMD 10 delivers cardioversion and/or defibrillation shocks to heart 16, and more particularly right ventricle 20 and left ventricle 22, via one or more of elongated coil electrodes 42 and 44, which may also be used in combination with housing electrode 46 in “active can” configurations of IMD 10.

IMD 10 detects a ventricular arrhythmia by sensing cardiac electrical signals using any of the electrodes 30, 32, 34, 36, 42, 44 and/or housing electrode 46. If a sufficient number of VCLs (the NID) fall within the VCL zone associated with a particular arrhythmia, the corresponding arrhythmia is detected. IMD 10 stores the NID for each arrhythmia. The NID can be expressed, for example, as a number of intervals falling within an arrhythmia VCL zone out of a given number of most recent cardiac intervals, e.g. 9 out of 12 of the most recent intervals may be required to have a VCL within a VF zone in order to detect VF. It is understood that other detection criteria may be implemented including criteria relating to the cardiac signal morphology or criteria relating to other physiological signals.

As will be described in greater detail below, IMD 10 may have multiple NID settings for a given arrhythmia zone. The NID used to detect an arrhythmia at any given time is selected based on information relating to patient physical activity. In some embodiments, for example, IMD 10 increases the NID when patient activity is stable or increasing. IMD 10 may decrease the NID if the patient activity is stable or decreasing.

The configuration of IMD 10 and leads 14 illustrated in FIG. 1 is merely illustrative. In various embodiments, IMD 10 is coupled to any number of leads that extend to a variety of positions within or outside of heart 16. For example, in some embodiments, IMD 10 is coupled to a lead 14 that extends to a position within coronary sinus and great vein 26 near left atrium 24 for left ventricular sensing and/or pacing. In some embodiments, at least some of leads 14 are epicardial leads. Further, in some embodiments, IMD 10 may utilize subcutaneous electrodes, incorporated along the housing of the IMD 10 for sensing cardiac electrical activity. A subcutaneous IMD system may include one or more electrodes carried by a leads extending to a location outside the thoracic cavity for sensing ECG signals.

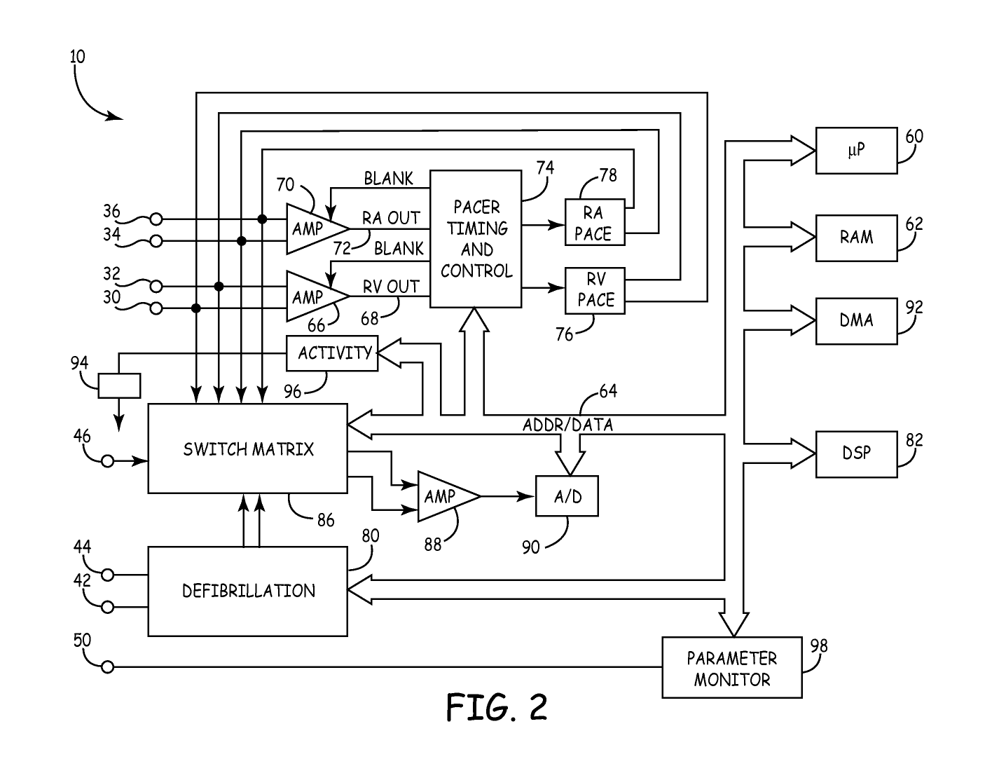

FIG. 2 is a functional block diagram of IMD 10. In the illustrated embodiment, IMD 10 takes the form of a multi-chamber pacemaker with cardioversion and/or defibrillation capability having a microprocessor-based architecture. However, this diagram should be taken as illustrative of the type of device in which various embodiments of the methods described herein may be embodied, and not as limiting.

IMD 10 includes a microprocessor 60. Microprocessor 60 executes program instructions stored in memory, such as a ROM (not shown), EEPROM (not shown), and/or RAM 62, which control microprocessor 60 to perform the functions ascribed to microprocessor 60 herein. Microprocessor 60 is coupled to, e.g., communicates with and/or controls, various other components of IMD 10 via an address/data bus 64.

Electrodes 30 and 32 are coupled to amplifier 66, which takes the form of an automatic gain controlled amplifier providing an adjustable sensing threshold as a function of the measured R-wave amplitude. A signal is generated on RV out line 68 whenever the signal sensed between electrodes 30 and 32 exceeds the present sensing threshold. Electrodes 34 and 36 are coupled to amplifier 70, which also takes the form of an automatic gain controlled amplifier providing an adjustable sensing threshold as a function of measured P-wave amplitude. A signal is generated on RA out line 72 whenever the signal sensed between electrodes 34 and 36 exceeds the present sensing threshold.

IMD 10 paces heart 16. Pacer timing/control circuitry 74 includes programmable digital counters which control the basic time intervals associated with modes of pacing. Pacer timing/control circuitry 74 also controls escape intervals associated with pacing. In a dual-chamber device, pacer timing/control circuitry 74 controls the atrial and ventricular escape intervals that are used to time pacing pulses delivered to right atrium 18 and right ventricle 20.

During pacing, escape interval counters within pacer timing/control circuitry 74 are reset upon sensing of R-waves and P-waves as indicated by signals on lines 68 and 72, and, in accordance with the selected mode of pacing, circuitry 74 controls generation and delivery of pacing pulses upon time-out of the escape intervals. Circuitry 74 also resets escape interval counters upon generation of pacing pulses. Circuitry 74 uses the value of the count present in escape interval counters when reset by sensed R-waves and P-waves, or delivered pacing pulses, to measure ventricular cycle lengths (VCLs) and, in some embodiments, atrial cycle lengths (ACLs). Circuitry 74 provides the measured VCL and ACL values to microprocessor 60 for detection of arrhythmias via address/data bus 64.

Microprocessor 60 operates as an interrupt driven device, and is responsive to interrupts from pacer timing/control circuitry 74 corresponding to the occurrence of sensed P-waves and R-waves and corresponding to the generation of cardiac pacing pulses. Those interrupts are provided via data/address bus 64. Any necessary mathematical calculations to be performed by microprocessor 60 and any updating of the values or intervals controlled by pacer timing/control circuitry 74 take place following such interrupts.

In accordance with the selected mode of pacing, pacer timing/control circuitry 74 triggers generation of pacing pulses by one or more of pacer output circuits 76 and 78, which are coupled to electrodes 30 and 32, and 34 and 36, respectively. Output circuits 76 and 78 are pulse generation circuits, which include capacitors and switches for the storage and delivery of energy as a pulse.

In some embodiments, IMD 10 detects ventricular arrhythmias using a tachycardia and fibrillation detection algorithm. For example, RAM 62 can store NID thresholds for various ventricular arrhythmias, and microprocessor 60 can detect ventricular arrhythmias by receiving VCLs from pacer timing/control circuitry 74, categorizing the VCLs based on VCL ranges for the various types of arrhythmias, and counting VCLs within each category. A clinician can program values for the VCL ranges and NIDs via a programming device and device telemetry. NIDs stored by RAM 62 can include one or more NID values for each arrhythmia detection zone including, for example, VF, VT, and fast VT (FVT), and a combined count NID in which intervals classified as VF and VT are considered together.

In response to detection of an arrhythmia, IMD 10 is capable of delivering one or more anti-tachycardia pacing (ATP) therapies to heart 16, and/or defibrillation or cardioversion shocks to heart 16 via one or more of electrodes 30-36 and 42-46. Electrodes 42 and 44 are coupled to defibrillation circuit 80, which delivers defibrillation and/or cardioversion pulses under the control of microprocessor 60. Defibrillation circuit 80 includes energy storage circuits such as capacitors, switches for coupling the storage circuits to coil electrodes 42 and 44, and logic for controlling the coupling of the storage circuits to the electrodes to create pulses with desired polarities and shapes. Microprocessor 60 may employ an escape interval counter to control timing of such defibrillation pulses, as well as associated refractory periods.

A clinician can program therapies or sequences of therapies for each of the types of ventricular arrhythmias via a programming device and device telemetry, and information describing the programmed therapies and/or sequences of therapies can be stored in RAM 62. The programmed therapies can include sequences of one or more defibrillation pulses, one or more cardioversion pulses, one or more ATP attempts, or combinations thereof.

In some embodiments, a ventricular electrogram (EGM) signal received via a combination of any of electrodes 30, 32 and 46 is digitally processed by a digital signal processor (DSP) 82 for morphological analysis of the signal. In such embodiments, switch matrix 86 is used to select which of electrodes 30, 32 and 46 are coupled to amplifier 88 for use in digital signal analysis. Selection of electrodes is controlled by microprocessor 60 via data/address bus 64, and the selections may be varied as desired. The EGM signal is converted to a digital signal by analog to digital converter circuit 90, and provided to DSP 82 via address/data bus 64. In some embodiments, the digital signal is stored in RAM 62 under control of direct memory access (DMA) circuit 92, and retrieved therefrom by DSP 82 for analysis. In other embodiments, IMD 10 does not include a dedicated DSP 82, and microprocessor 60 performs digital signal analysis.

IMD 10 is shown in FIG. 2 as including an activity sensor 94. Microprocessor 60 determines which NID values are used for detecting an arrhythmia at any given time based on the activity level of the patient. Activity sensor 94 provides a sensor output that varies as a function of the level of physical activity of the patient. In some embodiments, activity sensor 94 is a piezoceramic accelerometer bonded to a hybrid circuit located inside housing 48 (shown in FIG. 1). An accelerometer-based activity sensor may alternatively be located outside the IMD housing, e.g. carried by a lead or implemented as a leadless sensor in telemetric communication with IMD 10. In other embodiments, activity sensor 94 additionally or alternatively comprises electrodes to detect respiration rate via cyclical variations in the thoracic impedance.

The output signal provided by activity sensor 94 is coupled to an activity detection circuit 96, which determines the activity level of the patient based on the output. An activity level count (ALC) may be determined from an accelerometer signal and updated according to numerous possible algorithms. In one embodiment, a weighted ALC is acquired over a six second interval which is updated every 2 seconds. The ALC may be obtained according to methods generally described in U.S. Pat. No. 6,449,508 (Sheldon, et. al), hereby incorporated herein by reference in its entirety. Updating of the ALC may be enabled to occur continuously beginning from the time of device implant such that ALCs can be updated throughout time.

In the illustrated example, IMD 10 also includes a parameter monitor circuit 98. Parameter monitor circuit 98 processes the signal received from a physiological sensor 50, and provides a result of the processing to microprocessor 60 for use in detecting arrhythmias. Sensor 50 may be a pressure sensor, acoustical sensor, blood oxygen sensor or other type of sensor that provides additional physiological information for use by IMD 10 in detecting arrhythmias.

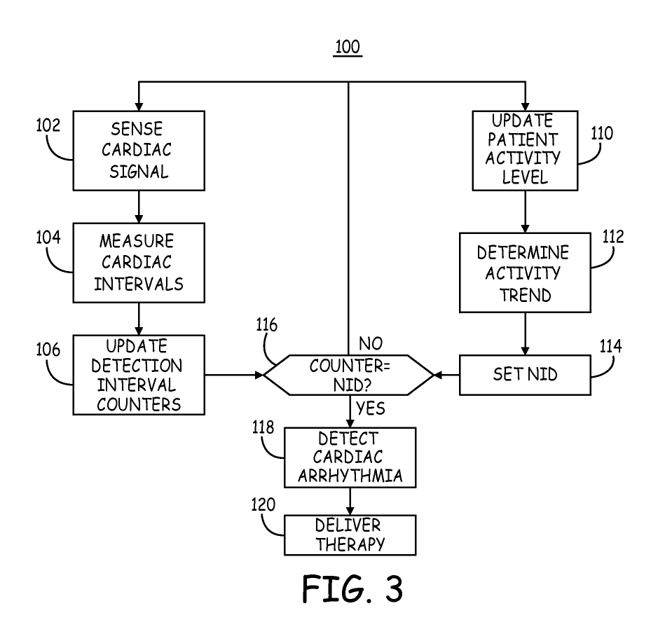

FIG. 3 is a flow chart 100 of a method for controlling arrhythmia detection based on patient activity. Flow chart 100 is intended to illustrate the functional operation of the device, and should not be construed as reflective of a specific form of software or hardware necessary to practice the methods described. It is believed that the particular form of software will be determined primarily by the particular system architecture employed in the device and by the particular detection and therapy delivery methodologies employed by the device. Providing software to accomplish the described functionality in the context of any modern IMD, given the disclosure herein, is within the abilities of one of skill in the art.

Methods described in conjunction with flow charts presented herein may be implemented in a computer-readable medium that includes instructions for causing a programmable processor to carry out the methods described. A “computer-readable medium” includes but is not limited to any volatile or non-volatile media, such as a RAM, ROM, CD-ROM, NVRAM, EEPROM, flash memory, and the like. The instructions may be implemented as one or more software modules, which may be executed by themselves or in combination with other software.

At block 102, one or more cardiac signals are sensed using available electrodes as described above for measuring cardiac intervals, e.g. VCLs, at block 104. At block 106, a detection interval counter is updated when a measured cardiac cycle length falls within a detection zone. Multiple detection interval counters may be used for detecting different arrhythmia types, e.g. a VF interval counter, VT interval counter, fast VT interval counter, combined count, and so on. If a counter is increased in response to a measured cardiac cycle length, the counter value is compared to the NID currently set for detecting the corresponding arrhythmia type at decision block 116.

As cardiac signal monitoring is occurring at blocks 102 through 106, patient activity is being monitored at blocks 110 through 114 for use by the IMD microprocessor in setting the NID that is used at decision block 116. At block 110, the patient activity level is updated according to an activity monitoring algorithm. The patient activity level may be an activity level count that is updated every few seconds, e.g. approximately every 1 to 10 seconds, though longer update intervals could also be used. In general, a patient activity update interval applied at block 110 will be less than approximately one minute in order to provide a relevant measure of the patient's current activity level for setting NID values. In one embodiment, an ALC is updated every 6 seconds.

At block 112, the updated patient activity level is compared to a previous activity level measurement to determine a patient activity trend. The most recent activity level measurement may be compared to the currently updated activity level measurement to determine if the patient's activity is remaining substantially the same (e.g. within a predetermined range of the previous measurement), is increasing or is decreasing. The activity trend may alternatively be determined through other trending techniques such as linear and non-linear regressions and digital filtering. The patient activity trend is used to set the NID value for one or more arrhythmia detection zones at block 114.

The NID may be kept the same, increased or decreased for one or more arrhythmia detection zones at block 114. In one embodiment, the NID for detecting shockable rhythms, i.e. VF and unstable VT, is increased in response to a steady or increasing activity level trend and decreased in response to a decreasing activity level trend. A clinician may program a default NID value and at least one higher NID value to be used when patient activity is steady at a moderate or high level or patient activity is increasing. The NID may be adjusted between the default value (e.g., when a decreasing activity level is detected) and the higher value (e.g., when patient activity is steady or increasing). Alternatively, in response to a steady activity level, no adjustment to an NID value is made at block 114. The NID is decreased to a default value in response to a decreasing activity trend and increased to a higher value in response to an increasing activity trend.

After selecting the NID value for one or more detection zones based on the patient activity level trend at block 114, the current detection interval counter values are compared to the selected NID value(s) at block 116. A cardiac arrhythmia is detected at block 118 when a detection interval counter reaches a currently selected NID value. An arrhythmia therapy may be delivered according to programmed therapy delivery algorithms as indicated at block 120.

If none of the detection interval counters meet the currently selected NID value(s) at block 116, the process returns to blocks 102 and 110 to continue to simultaneously monitor the cardiac cycle intervals for updating detection interval counters and monitor patient activity for updating NID in accordance with monitored patient activity. In this way, if a patient is sustaining a moderate or high level of activity or increasing his/her activity, a fast heart rate will be less likely detected as a shockable rhythm. A shockable rhythm is considered a potentially lethal rhythm, or at least a hemodynamically compromised rhythm, in which the patient would not likely be able to sustain or increase his/her activity. If the patient's activity is decreasing when a fast rate is detected, the decrease in patient activity provides supporting evidence that the patient may be hemodynamically compromised. Detection of the fast rate as a treatable arrhythmia may proceed according to nominal or default detection parameters.

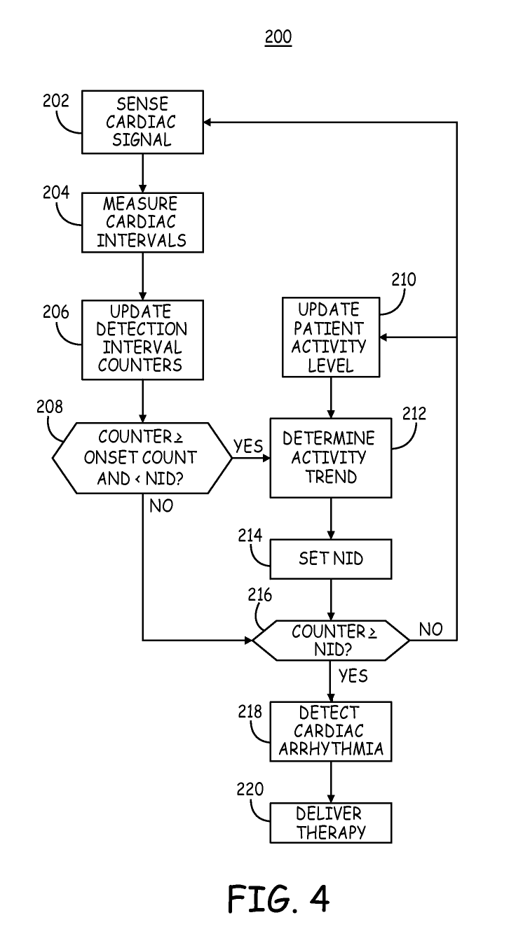

FIG. 4 is a flow chart 200 of an alternative method for controlling arrhythmia detection parameters based on patient activity. At block 202, one or more cardiac signals are sensed using available electrodes as described above for measuring cardiac intervals at block 204. At block 206, a detection interval counter is updated when a measured cardiac cycle length falls within a detection interval zone. If a counter is updated in response to a measured cardiac cycle length, the counter value is compared to an onset threshold at decision block 208. The onset threshold is a lower number of intervals than the NID set for detecting an arrhythmia.

For example, in one embodiment, the onset threshold is set as two consecutive intervals falling into a tachycardia or fibrillation rate zone. If two consecutive intervals are shorter than a VT or VF maximum interval, the onset threshold is met. The onset threshold may vary between embodiments to be any number of intervals less than the NID, and may require consecutive or non-consecutive intervals that fall within VT or VF interval ranges.

In alternative embodiments, the onset threshold may relate to the same or a different detection parameter than the one being adjusted in response to patient activity analysis. The onset threshold may be applied to an interval or a morphology based metric useful in detecting or discriminating arrhythmias.

In response to the onset threshold being reached at block 208, the patient activity trend is updated at block 212 based on the most recently updated patient activity level measured at block 210. NID is then adjusted at block 214 using the activity trend as described above or a combination of the current activity level and the activity trend. As such, during cardiac signal monitoring at block 202 through 208, patient activity is updated regularly at block 210 for use by the IMD microprocessor in updating a trend and setting the NID if an onset threshold is reached.

The NID may be kept the same, increased or decreased for one or more arrhythmia detection zones at block 214. After selecting the NID value for one or more detection zones based on the patient activity level trend at block 214, the current detection interval counter values are compared to the selected NID value(s) at block 216. A cardiac arrhythmia is detected at block 218 when a detection interval counter reaches the currently set NID value. An arrhythmia therapy may be delivered according to programmed therapy delivery algorithms as indicated at block 220.

If none of the detection interval counters meet the currently selected NID value(s) at block 216, the process returns to blocks 202 and 210 to continue to simultaneously monitoring the cardiac cycle intervals for updating detection interval counters and monitor patient activity for updating NID values.

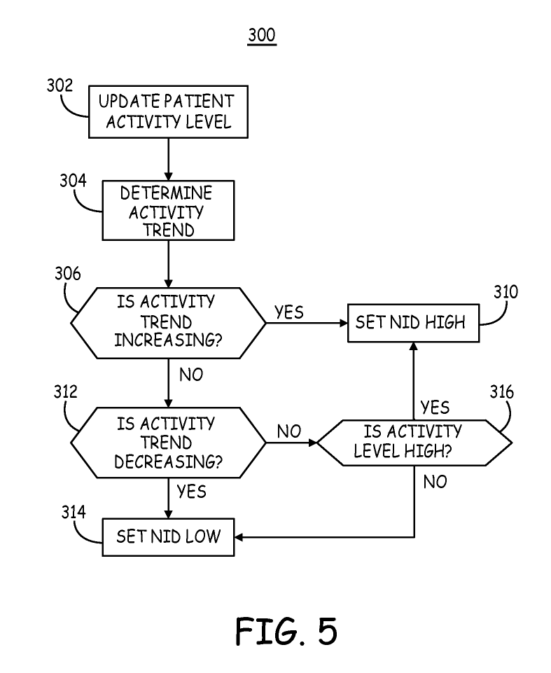

FIG. 5 is a flow chart of one method for adjusting NID in response to a patient's activity level and activity trend. While FIG. 5 and other embodiments described herein refer particularly to adjusting NID, it is to be understood that other arrhythmia detection parameters, such as detecting interval zones, morphology-related parameters, or other interval-related parameters, may be adjusted in response to an analysis of the patient activity level and the patient activity trend. The arrhythmia detection parameters defined for detecting an arrhythmia may vary between embodiments and which parameters are adjusted in response to activity level and activity trend may be tailored to a particular detection algorithm and patient.

At block 302, the patient activity level is updated using the activity sensor signal, e.g. using activity level counts as described in the above-incorporated '508 patent. The activity level is updated periodically, for example at least once per minute and in some embodiments every several seconds.

The process shown in flow chart 300 may be performed continuously such that the NID (or other detection parameter) is being adjusted as patient activity changes independent of arrhythmia detection counter values or other rhythm status indicators. Alternatively, patient activity level is regularly updated at block 302, but the activity level data is not used to adjust NID until hen an onset threshold has been met as described in conjunction with FIG. 4.

At block 304, the activity level trend is determined in response to the updated patient activity level. The activity trend may be determined by comparing the current activity level to a prior activity level. In one embodiment, an activity level count (ALC) is updated every two seconds using weighted sums of the latest three, two-second activity level counts. The updated ALC may be compared to a prior ALC obtained six seconds earlier (having no overlapping two-second activity count intervals with the currently updated ALC). If the updated ALC is greater than the earlier ALC, an increasing trend in activity is detected; if less than the earlier ALC, a decreasing trend is detected. If the updated and earlier ALCs are approximately equal, the activity trend is considered flat or substantially unchanged. Numerous variations may be used for detecting an activity level trend, which will depend in part on what type of activity sensor is being used and the algorithm used to detect patient activity level.

Methods described herein for adjusting NID are not limited to one particular method for determining a patient activity trend. For example, longer or shorter time periods may be used for determining an updated activity level and a prior activity level for determining a trend. An average, mean, median, cumulative differences, or other metric of ALCs over a period between approximately two seconds and up to one minute or more may be determined as an updated ALC. A prior activity level may be computed over a previous interval of two or more seconds up to approximately five minutes.

The activity trend may alternatively be determined through trending techniques such as linear and non-linear regressions, digital filtering and cumulative differences. In case of linear regression, the rate of change of ALC may be computed over multiple intervals during the most recent time period, e.g. up to five minutes. If the rate of change of ALC in the most recent interval, e.g. an interval of approximately 6 seconds up to 1 minute, exceeds the range of rates observed during a prior time period, e.g. a prior interval of 6 seconds up to 5 minutes, a predetermined number of times, an increasing trend may be determined.

In case of non-linear regression analysis, a quadratic or a higher order polynomial trend of ALC may be computed over a prior period ranging from 6 seconds up to approximately 5 minutes in duration. The rate of change of ALC, based on such trend, may be compared between a most recent interval (e.g. six seconds up to approximately one minute) and one or more earlier intervals of the same or varying durations.

If cumulative differences in activity level are computed, the cumulative difference may be compared to a threshold to detect an activity trend. Alternatively the trend of the ALC may be computed using a moving average of cumulative differences, non-linear regression or digital filtering of cumulative differences over a period of approximately 6 seconds up to 5 minutes.

If the activity trend is increasing, the NID is adjusted to a higher value at block 310. The actual patient activity level as updated at block 302 may be low, moderate or high. If the activity trend is increasing as determined at decision block 306, the patient is presumed to be hemodynamically stable and the NID is set high to reduce the likelihood of a false detection of a shockable rhythm. The patient's heart rate may be increased and presenting a normal sinus tachycardia response to increased activity. The increasing activity trend presents evidence contradictory to a hemodynamically unstable heart rhythm. The NID is adjusted based on the increasing activity trend, independent of the actual activity level.

If the trend is not increasing, the process advances to decision block 312 to determine if the activity trend is decreasing. If the activity trend is decreasing, the NID is adjusted to a lower (or nominal) setting at block 314. The actual activity level most recently updated at block 302 may be low, high or moderate, but if activity is decreasing, this decreasing activity does not provide evidence that would contradict the detection of a hemodynamically unstable heart rhythm. Even if the patient's current activity is still high or moderate, the decreasing trend may indicate that the patient is unable to sustain the activity due to deteriorating heart rhythm and hemodynamics.

If the activity trend is neither increasing nor decreasing (negative results at both decision block 306 and 312), the updated activity level is used as the basis for setting the NID. In other words, if the activity trend is substantially flat or unchanging, indicating a sustained activity level, the trend information is inconclusive as to whether the patient may be hemodynamically stable or not. Instead, the actual activity level provides is used to set the arrhythmia detection parameter(s).

If the activity level is high and not changing as determined at decision block 316, the NID is set to a high setting at block 310. A high, sustained level of activity suggests the patient is hemodynamically stable. More stringent criteria for detecting an arrhythmia is used; more stringent criteria results in an arrhythmia detection requiring more time or more cardiac cycles to detect an arrhythmia episode.

If the activity level is low at decision block 316, the NID is set to a low (or nominal) value at block 314. A sustained low level of activity does not provide evidence that would contradict an arrhythmia detection. As such, arrhythmia detection criteria remains at nominal or default settings to allow arrhythmia detection to occur relatively more quickly than when a high activity level with either a sustained or increasing trend is detected.

By using a combination of both activity trend and the current activity level, situations where the activity level is low but increasing or high but decreasing can be used to appropriately adjust an NID to a higher level or a lower level, respectively, when patient activity provides evidence that the patient is either hemodynamically stable in the first case or potentially unstable in the latter case. If activity level was used alone without considering the trend in activity, a low level of activity may result in a low NID setting even when the patient activity is beginning to increase, suggesting that an increased heart rate is an appropriate sinus tachycardia response. The low NID setting could result in a false arrhythmia detection. If a high activity level is used to set NID high, but the activity level is falling due to hemodynamic insufficiency, the high NID may delay an appropriate arrhythmia detection.

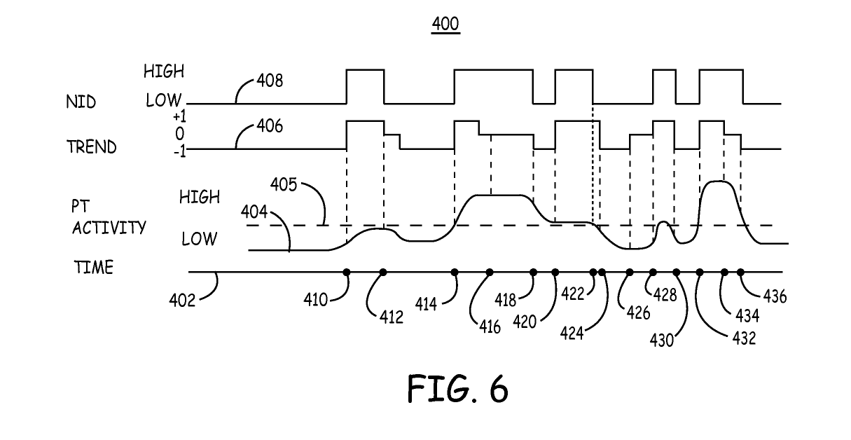

FIG. 6 is a time line 400 illustrating the behavior of an arrhythmia detection parameter over time as patient activity changes. Activity level 404, activity level trend 406 and NID 408 are plotted over time 402. High and low activity levels may be distinguished by a threshold 405. In alternative embodiments, multiple threshold levels may be defined to separate multiple activity ranges, e.g. a low, moderate and high activity level. Multiple arrhythmia detection parameter settings may be associated with the various activity level ranges.

The activity trend 406 is shown to vary between a decreasing trend (−1), sustained or flat trend (0), and an increasing trend (+1). Changes between decreasing, sustained and increasing trend levels may be delayed from an actual change observed in the activity level 404. The detected activity trend 406 may lag an actual change in activity level 404 due to the time period required for updating the activity level to obtain consecutive measurements that are compared to determine the trend. The lag between detecting an activity trend and an actual change in the activity level may also include a required time interval during which a monotonic change is required in order to verify a detected trend. For example, an increasing, decreasing, or sustained trend may be detected only when a required number of consecutive updated activity levels show a consistently increased, decreased, or sustained activity level, respectively.

NID 408 is shown to vary between high and low levels. It is recognized that in alternative embodiments, multiple settings may be applied to an arrhythmia detection parameter in response to different rates of activity level change, activity level, or a combination of both. Two settings are shown here for the sake of clarity. In one embodiment, a low setting may be a nominal setting of, for example 9 out of the most recent 12 RR intervals. The high setting may require a higher number out of the same number of RR intervals (e.g. 12 out of 12) or a higher number out of a greater number of RR intervals (e.g. 18 out of 24 RR intervals), which may be the same or a different ratio of detection intervals to total number of intervals. The low and high settings may be programmable and selected according to clinician preference.

At time point 410, activity is increasing from an initially low level. The activity trend is detected as increasing (+1). In response to an increasing activity trend, NID 408 is increased to a high level. The actually activity level remains relatively low, below a threshold 405 defined to separate high and low activity levels, but NID is increased in response to the increasing activity trend.

At time point 412, the activity trend is found to be relatively flat (0). For The updated activity level has remained within a percentage range of a previous activity level. The actual activity level remains low (below threshold 405). In response to a flat activity trend and low activity level, NID is decreased at time point 412 back to a low value.

At time point 414, an increasing activity trend causes NID to increase again. This time, however, detection of a flat or sustained activity trend at time point 416 causes the NID to remain at the high level because the activity level 404 is high (above threshold 405).

At time point 418, the activity trend is decreasing. Even though activity 404 is still high (greater than threshold 405), the decreasing trend causes NID 408 to be adjusted to the low level.

The activity level 404 plateaus resulting in a flat activity trend being detected at time point 420. A flat activity trend with an actual activity level still high (above threshold 405) causes NID to return to the high setting at time point 420.

When activity decreases to below the activity threshold 405, the NID 408 may be immediately adjusted to the low setting at time point 422. Alternatively, NID 408 may not be adjusted to the low setting until a decreasing trend is detected at block 424.

At time point 426, the activity level remains low and the trend is substantially flat so the NID remains low. At time point 428, the activity level is still low but the increasing trend causes NID to be increased. The decreasing trend at time point 430 causes NID to be decreased again. In other embodiments, a larger lag time for detecting an increasing or decreasing trend in activity may be required to avoid frequent adjustments of NID in response to short bouts of increased activity. Short spikes in the activity level signal could be caused by noise or artifact affecting the activity sensor which should not cause frequent changes in the NID setting.

At time point 432, an increased trend is detected causing NID to go to a high setting. The sustained trend detected at block 434 results in a sustained high setting of NID because the activity level 404 remains high (above threshold 405). At time point 436, activity 404 is still high but the NID is decreased to a low setting in response to a decreasing activity trend being detected.

As shown in FIG. 6, the NID setting may be adjusted in response to a combination of both activity level and trend. In some cases NID is adjusted based on the activity level (when the trend is flat or sustained) and in other cases NID is adjusted based on the activity trend (when increasing or decreasing) independent of the actual activity level. The NID may be adjusted as shown in FIG. 6 independent of the value of any arrhythmia detection counters. In other words the behavior shown may be occurring even when detection counters have a zero value. This allows a sudden increase in a counter value, e.g. due to non-physiologic noise or other causes, to use an appropriate NID that has already been established based on updated activity level and trend.

In other embodiments, NID is not adjusted at all unless a detection counter has reached an onset threshold as described above. Although a detection counter is not shown in FIG. 6, it can be appreciated that the NID may remain at a low level or nominal setting independent of changes in activity level and activity trend unless a counter has reached an onset threshold. If the onset threshold is reached but a detection threshold has not been reached, the NID will be controlled based on activity level and activity trend as shown in FIG. 6 until a detection is made or until the counter is reset to a zero value or falls below the onset threshold.

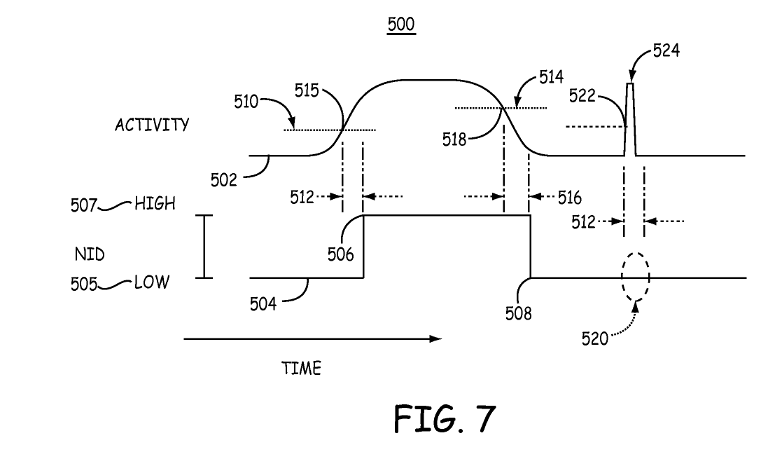

FIG. 7 is a time line 500 depicting adjustment of NID based on a delay time following detection of an increasing activity. Activity 502 and NID 504 are shown over time. A threshold 510 is defined for detecting an increasing trend as activity rises from an initially low level to a higher level. The positive-going threshold crossing at 515 results in a detection of an increasing activity trend. NID will not be adjusted immediately, however. NID will be adjusted to a higher setting 507 at time point 506 after a time delay 512 has expired, as long as the activity level has remained above threshold 510 for the entire delay time 512.

A second threshold 514 is defined for detecting a decreasing activity trend. The second threshold 514 may be defined to be a different value, e.g. a higher value, than the first threshold 510. If the activity level remains below the second threshold 514 for at least a time delay 516 after the negative-going threshold crossing at 518, NID will be decreased to the lower setting 505 at time point 508 upon expiration of the time delay 516. The time delay 516 applied to the detection of a decreasing activity trend may be defined uniquely from the time delay 512 applied to the detection of an increasing activity trend.

A short pulse 524 of increased activity does not result in adjustment of NID as highlighted by dashed circle 520. While a positive-going threshold crossing is detected at 522, the activity signal falls below threshold 510 before expiration of the adjustment delay 512. The delay intervals 512 and 516 prevent frequent adjustment of NID in response to short fluctuations in activity level.

In addition or alternatively to using actual activity level during periods of unchanging or flat activity trend, the NID may be adjusted based on the patient posture, e.g., supine, upright or laterally recumbent. The posture may be determined based on a 3-axis accelerometer 94. Following a predetermined period of inactivity, such as an ALC of substantially zero or a predetermined level less than threshold 510, and in the absence of any VF or VT detection by the IMD, NID may be adjusted, e.g. increased to a predetermined level between 505 and 507, for supine and laterally recumbent patients.

The thresholds 510 and 514 may be auto-adjusting thresholds based on a percentage or range of a previously measured activity level. For example, threshold 510 may be set as a percentage of an initial low level of activity, a baseline activity, or a minimum activity reached after a negative-going threshold crossing. Threshold 514 may be set as a percentage of the maximum activity level reached after a positive-going threshold crossing 515.

Thus, an implantable medical device and associated method for optimizing arrhythmia detection using patient physical activity have been presented in the foregoing description with reference to specific embodiments. It is appreciated that various modifications to the referenced embodiments may be made without departing from the scope of the disclosure as set forth in the following claims.

1. A method for detecting cardiac events for use in an implantable medical device, comprising:
sensing a patient activity signal;
determining a patient activity trend in response to the activity signal;
setting a cardiac event detection threshold in response to the patient activity trend;
sensing a cardiac signal; and
detecting a cardiac event in response to the cardiac signal and the detection threshold.

2. The method of claim 1, wherein the detection threshold comprises a number of cardiac intervals required to detect the cardiac event.

3. The method of claim 1, wherein sensing the activity signal comprises sensing an accelerometer signal, the accelerometer positioned in a housing of the implantable medical device.

4. The method of claim 1, further comprising:
establishing an activity threshold;
detecting a threshold crossing of the activity signal;
starting a delay interval in response to the threshold crossing; and
setting the cardiac event detection threshold upon expiration of the delay interval if the activity signal does not cross the threshold again before expiration of the delay interval.

5. The method of claim 1, further comprising:
establishing a cardiac event onset threshold relating to one of a cardiac interval and a cardiac signal morphology;
determining whether the onset threshold has been met; and
determining the patient activity trend and setting the detection threshold in response to the onset threshold being met.

6. The method of claim 1, further comprising establishing a first detection threshold value and a second detection threshold value, the first detection threshold value resulting in an earlier cardiac event detection than the second detection threshold value;
adjusting the detection threshold to the first detection threshold value in response to determining a decreasing patient activity trend; and
adjusting the detection threshold to the second detection threshold value in response to determining an increasing trend in patient activity.

7. The method of claim 1, wherein determining the patient activity trend comprises determining an updated activity level,
the detection threshold being set based on the updated activity level if the patient activity trend is a substantially unchanging patient activity.

8. The method of claim 1, further comprising detecting a patient posture,
the detection threshold being set in response to the patient posture in response to the patient activity trend being a substantially unchanging patient activity.

9. An implantable medical device for detecting cardiac events, comprising:
an activity sensor to sense a patient activity signal;
at least one electrode pair to sense a cardiac signal; and
a processor configured to determine a patient activity trend in response to the activity signal and set a cardiac event detection threshold in response to the patient activity trend, and detect a cardiac event in response to the cardiac signal and the detection threshold.

10. The device of claim 9, wherein the detection threshold comprises a number of cardiac intervals required to detect the cardiac event.

11. The device of claim 9, further comprising a housing enclosing the processor and the activity sensor, the activity sensor comprising an accelerometer.

12. The device of claim 9, further comprising a memory storing an activity threshold, wherein the processor is further configured to detect a threshold crossing of the activity signal, start a delay interval in response to the threshold crossing, and set the cardiac event detection threshold upon expiration of the delay interval if the activity signal does not cross the threshold again before expiration of the delay interval.

13. The device of claim 9, wherein the processor is further configured to establish a cardiac event onset threshold relating to one of a cardiac interval and a cardiac signal morphology, determine whether the onset threshold has been met, and determine the patient activity trend and set the detection threshold in response to the onset threshold being met.

14. The device of claim 9 further comprising a memory storing a first detection threshold value and a second detection threshold value, the first detection threshold value resulting in an earlier cardiac event detection than the second threshold value, wherein the processor is configured to adjust the detection threshold to the first detection threshold value in response to determining a decreasing patient activity trend and adjust the detection threshold to the second detection threshold value in response to determining an increasing trend in patient activity.

15. The device of claim 9, wherein the processor is further configured to determine an updated activity level and set the detection threshold based on the updated activity level if the patient activity trend is a substantially unchanging patient activity.

16. The device of claim 9, further comprising a posture sensor detecting a patient posture, wherein the processor is further configured to set the detection threshold in response to the patient posture in response to the patient activity trend being a substantially unchanging patient activity.

17. A computer-readable medium storing a set of instructions which cause a processor of an implantable medical device to:
sense a patient activity signal;
determine a patient activity trend in response to the activity signal;
set a cardiac event detection threshold in response to the patient activity trend;
sense a cardiac signal; and
detect a cardiac event in response to the cardiac signal and the detection threshold.
